(19) United States
(12) Patent Application Publication
(10) Pub. No.: US 2006/0218679 A1
(43) Pub. Date: Sep. 28, 2006

(54) Method of producing euphorbia interspecific hybrid plants by cutting and then culturing the hybrid embryos

(75) Inventor: Andrew Bernuetz, Silverdale (AU)

Correspondence Address: NIKOLAI & MERSEREAU, P.A., 900 SECOND AVENUE SOUTH, SUITE 820, MINNEAPOLIS, MN, 55402, US

(73) Assignee: Bonza Botanicals Pty Limited, Winmalee (AU)

(21) Appl. No.: 11/402,370

(22) Filed: Apr. 7, 2006

Related U.S. Application Data
(63) Continuation of application No. PCT/AU04/00257, filed on Feb. 27, 2003.

(30) Foreign Application Priority Data
Oct. 9, 2003 (AU) 2003905549

(51) Int. Cl. A01H 11/00

(52) U.S. Cl. 800/295; 47/58.1 R

(57) ABSTRACT

A method for producing an interspecific hybrid Euphorbia plant. The method comprises: (a) providing a first plant which is a Euphorbia pulcherrima plant and a second plant which is a species of Euphorbia selected from the group consisting of Euphorbia cornastra, Euphorbia radians, Euphorbia colorata and Euphorbia fulgens; (b) pollinating a flower of the second plant with pollen from the first plant or a flower of the first plant with pollen from the second plant in a manner which permits formation of an embryo in at least one ovule of the pollinated plant; (c) cutting the embryo; and (d) culturing the cut embryo by placing the cut embryo in contact with culture medium to permit growth of the embryo to thereby produce a primary plant.

### FIELD OF THE INVENTION

The present invention relates to Euphorbia interspecific hybrid plants and methods for making the same. In particular, the invention relates to interspecific hybrid plants derived from the cross between Euphorbia pulcherrima and species of Euphorbia other than Euphorbia pulcherrima, and methods for the generation of interspecific variants with altered characteristics to Euphorbia pulcherrima and other plants.

### BACKGROUND OF THE INVENTION

A characteristic of some plants is the ability to cross with different species, called interspecific hybridisation. This results in the transfer of genetic material between the species, leading to the production of an entirely new species of plant. Through the transfer of desirable genetic traits, it is possible to obtain improved species of plants which combine the desirable features of each of the parent plants. Interspecific hybridisation thus represents a method for producing new species of plants in which the desirable features of different species are combined.

However, not all species of plants are capable of undergoing interspecific hybridisation, and the ability of a plant to be hybridised with a different species can vary widely, depending on the species, the chromosome number of the plant, and the level of homology between the plant species to be crossed.

Interspecific hybridisation would be desirable between species within the genus Euphorbia. Euphorbia comprises a vast number of species. Among these, Euphorbia pulcherrima, also known as poinsettia, is among the most popular of ornamental potted plants. It would be desirable to be able to cross other species of Euphorbia with Euphorbia pulcherrima to retain the desirable characteristics of E. pulcherrima while also inheriting the desirable characteristics of other Euphorbia species. Features such as flowering period, flower colour, branch length, plant height, branch internode length etcetera may then be improved in the new species to provide a more desirable ornamental plant.

Methods for generating interspecific hybrids between Euphorbia species have met with limited success. For example, interspecific hybridisation has been possible to a limited extent between the poinsettia Euphorbia pulcherrima and Euphorbia cornastra as described in WO 02/32217. This document discloses an interspecific hybrid Euphorbia plant that was obtained using embryo rescue following pollination of Euphorbia pulcherrima with Euphorbia cornastra. Embryo rescue involves growing the embryo to a globular shaped stage of development following pollination, completely excising the embryo (and suspensor cells) from the ovary, and subsequently culturing the embryo in vitro. However, many of the embryos that are excised during embryo rescue do not survive, and/or do not grow into plants in culture. Further, generally it is not possible to produce more than one plant from a single embryo using embryo rescue. As a consequence, embryo rescue is often inefficient and/or unsuccessful for use in crosses between Euphorbia species.

### SUMMARY OF THE INVENTION

The inventor has found that by using a method in which the embryo is cut and the cut portions placed in culture, the efficiency of viable plants generated from an interspecific hybrid embryo developed by hybridising Euphorbia pulcherrima with other Euphorbia species can be improved.

In a first aspect, the invention provides a method for producing an interspecific hybrid Euphorbia plant comprising:

(a) providing a first plant which is a Euphorbia pulcherrima plant and a second plant which is a species of Euphorbia selected from the group consisting of Euphorbia cornastra, Euphorbia radians, Euphorbia colorata and Euphorbia fulgens;

(b) pollinating a flower of the second plant with pollen from the first plant or a flower of the first plant with pollen from the second plant in a manner which permits formation of an embryo in at least one ovule of the pollinated plant;

(c) cutting the embryo; and

(d) culturing the cut embryo by placing the cut embryo in contact with culture medium to permit growth of the embryo to thereby produce an interspecific hybrid Euphorbia plant (a primary plant).

Preferably, a flower of the first plant is pollinated with pollen from the second plant. In other words, preferably a flower of a Euphorbia pulcherrima plant is pollinated with pollen from a plant selected from the group consisting of Euphorbia cornastra, Euphorbia radians, Euphorbia colorata and Euphorbia fulgens.

The cutting step may comprise slicing the embryo into at least two portions. The term “cutting” includes slicing, splitting, breaking, or any other act that separates the embryo into at least two portions. Preferably, the portions are roughly equal portions.

In one embodiment, the embryo is cut while contained in the ovule. In a preferred embodiment, the ovule is at least 3 millimetres in length. The ovule may be sliced transverse to the longitudinal axis, or along the longitudinal axis. Preferably, the ovule is sliced along the longitudinal axis of the ovule.

Typically, the sliced ovule containing the sliced embryo is placed in contact with the culture medium.

Preferably, the culturing step employs ovule slice culture.

In another embodiment, the embryo is cut following excision from the ovule. For example, the embryo may be cut following embryo rescue. Preferably, the embryo is excised from an ovule that is at least 3 millimetres in length.

In one embodiment, the method comprises the further steps of:

(a) obtaining a cutting from the primary plant;

(b) incubating the cutting under conditions sufficient to propagate the primary plant.

Preferably, the cutting is a shoot.

The cutting may be treated to induce root formation. For example, root formation may be induced by treating the cutting with a composition containing a hormone capable of inducing root formation. Preferably, the hormone is an auxin. Examples of suitable auxins include indole-3-acetic acid (IAA), indole-3-butyric acid (IBA) and α-napthalene acetic acid (NAA).

A free-branching agent may be transmitted to the primary plant to provide the primary plant with a free-branching phenotype. Thus, in one embodiment, the method of the invention comprises the further step of transmitting a free-branching agent to the primary plant. The free-branching agent may be transmitted to the primary plant by any means. For example, the free branching agent may be transmitted to the primary plant by a dodder (a parasitic plant), by a leaf hopper insect or by a graft.

Preferably, the free-branching agent is transmitted to the primary plant by:

(a) providing a free-branching plant having a free-branching agent;

(b) cutting the primary plant and the free-branching plant to expose tissue of the plant;

(c) making a graft union between the tissue of the free-branching plant and the primary plant, whereby at least one characteristic of a vegetative shoot arising from said graft is different from the free-branching plant and the primary plant; and

(d) growing said shoot to obtain a grafted primary plant with at least one altered growth characteristic.

The grafted primary plant preferably differs from the primary plant in that the grafted primary plant has a free-branching phenotype. Thus, at least one altered growth characteristic is preferably a free-branching phenotype. Preferably, the free-branching phenotype is due to the free-branching agent.

The free-branching agent may be any agent which induces a free-branching phenotype and which can be transmitted by a graft, such as a bacterium, virus or phytoplasma. Preferably, the free-branching agent is a phytoplasma.

Variations in the characteristics of the grafted primary plant may be introduced by mutagenesis of the plant or parts thereof. In one embodiment, the method further comprises the steps of:

(a) obtaining a cutting of the primary plant or the grafted primary plant;

(b) exposing the cutting to a mutagen; and

(c) cultivating the cutting to produce a mutated plant.

The mutagen may be any mutagen capable of mutating plant DNA. The mutagen may be a chemical mutagen such as ethylmethane sulfonate, sodium azide, N-nitroso-N-ethylurea or N-nitroso-N-methylurea, a biological mutagen such as a transposon, or radiation. In one embodiment, the mutagen is radiation. Preferably, the radiation is gamma radiation.

The mutated plant, or a portion of the mutated plant, may be propagated by:

(a) obtaining a bract from the mutated plant;

(b) placing the bract in a solution capable of disinfesting the bract;

(c) washing the bract; and

(d) cultivating the bract to thereby produce a propagated mutated plant.

Preferably, the solution capable of disinfesting the bract is bleach. The bleach (NaOCl) is preferably used at a concentration of between 1% and 3.

The primary plant, or a portion of the primary plant, may be propagated by:

(a) obtaining a bract from the primary plant;

(b) placing the bract in a solution capable of disinfesting the bract;

(c) washing the bract; and

(d) cultivating the bract to thereby propagate the primary plant.

Preferably, the solution capable of disinfesting the bract is bleach. The bleach (NaOCl) is preferably used at a concentration of between 1% and 3%.

In a second aspect, the invention provides a plant produced according to the method of the first aspect of the invention.

In a third aspect, the invention provides part of a plant including, for example, a flower, cutting, a pollen grain, an ovule, a cell, a seed or an embryo, produced according to the method of the first aspect of the invention.

The plant may be a Euphorbia plant that is clonally propagated.

In a fourth aspect, the invention provides a method for producing an interspecific hybrid Euphorbia plant comprising:

(a) providing a first plant which is a Euphorbia pulcherrima plant and a second plant which is a species of Euphorbia that is not Euphorbia pulcherrima;

(b) pollinating a flower of the second plant with pollen from the first plant or a flower of the first plant with pollen from the second plant in a manner which permits formation of an embryo in at least one ovule of the pollinated plant;

(c) cutting the embryo; and

(d) culturing the cut embryo by placing the cut embryo in contact-with culture medium to permit growth of the embryo to thereby produce a primary plant.

In a fifth aspect, the invention provides a plant produced according to the method of the fourth aspect of the invention.

In a sixth aspect, the invention provides part of a plant including, for example, a flower, cutting, a pollen grain, an ovule, a cell, a seed or an embryo, produced according to the method of the fourth aspect of the invention.

In a seventh aspect, the invention provides a method for propagating a Euphorbia plant comprising:

(a) obtaining a bract from the plant;

(b) placing the bract in a solution capable of disinfesting the bract;

(c) washing the bract; and

(d) cultivating the bract to thereby produce a propagated mutated plant.

Preferably, the solution capable of disinfesting the bract is bleach. The bleach (NaOCl) is preferably used at a concentration of between 1% and 3%.

In an eighth aspect, the invention provides a plant produced according to the method of the seventh aspect of the invention.

In a ninth aspect, the invention provides part of a plant including, for example, a flower, cutting, a pollen grain, an ovule, a cell, a seed or an embryo, produced according to the method of the seventh aspect of the invention.

### BRIEF DESCRIPTION OF THE DRAWING

FIG. 1 illustrates the plants resulting from an embodiment of the method of the invention. The parent plants Euphorbia pulcherrima (A) and Euphorbia cornastra (B) are shown, with various interspecific hybrids of these parent plants in between A and B illustrating the variation in length, leaf size and internodal distance.

### DETAILED DESCRIPTION OF THE INVENTION

Before the present methods are described, it is understood that this invention is not limited to the particular materials and methods described, as these may vary. It must be noted that as used herein, the singular forms “a,” “an,” and “the” include plural reference unless the context clearly dictates otherwise. Thus, for example, a reference to “plant” includes a plurality of such plants. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

Publications mentioned hereinafter are cited for the purpose of describing and disclosing the protocols, reagents and vectors which are reported in the publications and which might be used in connection with the invention.

The present invention provides a method of producing an interspecific hybrid formed by crossing Euphorbia pulcherrima and a species of Euphorbia selected from the group consisting of E. cornastra, E. radians, E. colorata and E. fulgens.

The inventor has found that by cutting the embryo formed from interspecific pollination, the efficiency of hybrid plant production in culture is improved, and it is often possible to obtain multiple clonal interspecific hybrid plants from a single original embryo. This has advantages over a method such that described in WO 02/32217 because the present method allows more than one plant to be obtained from the same ovule or embryo. As a consequence, efficiency of interspecific hybrid production may be improved using the method of the invention. This is a surprising result because it was thought that the embryo produced by Euphorbia species was fragile and consequently would not survive disruption such as cutting of the embryo. Further, it was thought that an embryo from a Euphorbia interspecific hybrid was likely to be even more fragile than an embryo from an intraspecific hybrid. It was thought that cutting the Euphorbia embryo would not only damage the fragile tissue of the embryo and prevent normal function, but also that cutting a Euphorbia embryo would disrupt normal development of the embryo resulting in death of the embryo.

In a preferred embodiment, the first plant is a Euphorbia pulcherrima plant and the second plant is selected from the group consisting of Euphorbia cornastra, Euphorbia radians, Euphorbia colorata and Euphorbia fulgens.

The Euphorbia pulcherrima plant may be any cultivar of Euphorbia pulcherrima. Examples of suitable cultivars of Euphorbia pulcherrima include, for example, varieties such as 97/176.2, 97/176.3, 97/24.1, 97/54.1, 97/144, and cultivars such as Angelika (U.S. Plant Pat. No. 5,492), Freedom red (U.S. Plant Pat. No. 7,825) and V10 Amy red. The Euphorbia pulcherrima plant may be the derivative of a cross between two different Euphorbia pulcherrima cultivars. For example, cultivar 97/24.1 is the product of a cross between Euphorbia pulcherrima cv. Freedom white (U.S. Plant Pat. No. 8,772) and cv. V10 Amy red, cultivar 97/54.1 is the result of a self pollination of cv. V10 Amy red, 97/144 is the result of a cross between cv. Peppermint pink and cv. V1o Amy red.

As a first step in carrying out the method of the invention, the first and second plants are cultivated. As used herein, the term “cultivating” means to expose a plant to conditions which result in production of pollen from the first plant and presentation of a receptive stigma from the second plant, or in the production of pollen in the second plant and the presentation of a receptive stigma from the first plant. Preferably, the conditions result in production of a flower in the first and second plants. Methods for cultivation of Euphorbia species are well-known in the art and are described in, for example, Ecke, P., Matkin, O. A. and Hartley, D. E. (1990) The Poinsettia Manual. Paul Ecke Poinsettias, Encinitas, Calif., USA. It will be appreciated by those skilled in the art that growth conditions for the various Euphorbia plants used in the method of the invention may vary from species to species, and may depend on growth requirements such as photoperiod for each individual species.

The first or second plant may be emasculated to avoid self-pollination. This may be achieved by manual removal of the anthers, or by chemical means using gametocides such as described in U.S. Pat. No. 4,936,904.

The flower of the first plant is then pollinated with pollen from the second plant, or the flower of the second plant is pollinated with pollen from the first plant. Preferably, the flower of the first plant is pollinated with the pollen from the second plant. In other words, in a preferred embodiment, Euphorbia pulcherrima is pollinated with pollen from Euphorbia cornastra, Euphorbia radians, Euphorbia colorata or Euphorbia fulgens. As used herein, the term “pollinating” refers to any method by which pollen is brought into contact with the stigma of a plant in a manner which results in formation of an embryo in at least one ovule of the plant. In the case of interspecific hybridisation, the pollen from one species of Euphorbia is used to pollinate a different species of Euphorbia. Pollinating may be conducted manually using implements such as paint brushes or other methods known in the art for pollinating plants (see, for example, Watts, L. (1980) Flower and Vegetable Plant Breeding. Grower Books, London; Allard, R. W. (1999) Principles of Plant Breeding, John Wiley & Sons, incorporated herein by reference). It will be appreciated by those skilled in the art that the time for pollinating will depend on flowering times of the plant, and rate of production of anthers and pistils. It will also be appreciated by those skilled in the art that the times for flowering and obtaining pollen will vary from species to species and may be determined empirically using approaches well known in the art. Preferably, fresh pollen is collected and applied liberally to all areas of the receptive stigmatic surface.

Once the plant is pollinated, embryo development is detected, the embryo is cut, and the cut embryos cultured. Embryo development is preferably detected by observing cyathia with swollen ovaries. The embryo is then cultured from those ovaries that are swollen. Preferably, the stage at which the embryo is cultured is when an abscission layer is formed on the cyathium pedicel. As used herein, the term “cultured” refers to the process by which one or more embryos is grown in vitro, or in other words, in culture. The cultured embryo may be in contact with the ovule, or may be free of the ovule.

In one embodiment, the embryo is cut by removing the ovules from the swollen ovaries, and thereafter slicing the ovule in a manner which cuts the embryo. The ovule may be sliced in any direction which results in cutting of the embryo. For example, the ovule may be sliced along the longitudinal axis, transverse of the longitudinal axis or diagonal to the longitudinal axis. The ovule is preferably sliced along the longitudinal axis to thereby cut the embryo along its longitudinal axis. The ovule and embryo is then placed in culture medium. The ovule may be placed in any manner which permits growth of the embryo in the culture medium. Preferably, the cut ovule is placed in culture medium with the cut portion facing upwards.

In another embodiment, the embryo is extracted from the ovule and the embryo is subsequently cut prior to placing the cut embryo in culture medium.

The culture medium may be any medium that permits growth of the embryo in a manner that results in plantlets being generated from the embryo. For example, the culture medium may be tissue culture medium such as that described in, for example, Murashige, T and Skoog, F (1962). A revised medium for rapid growth and bioassays with tobacco tissue cultures. Physiologica Plantarum 15:473-497. Suitable culture mediums may include regeneration medium. An example of regeneration medium includes Murashige and Skoog (MS) salts (Sigma-Aldrich Pty. Ltd. catalogue number M5519) at 4.42 g/L activated charcoal preferably at 1 g/L, casein hydrolysate preferably at 1 g/L, sucrose preferably at 40 g/L and agar preferably at 7 g/L. pH is preferably adjusted to 5.8. The cultured embryo preferably is subcultured every 3 to 4 weeks. The embryo may be subcultured into fresh regeneration medium or may be subcultured into proliferation medium. An example of suitable proliferation medium includes MS salts at 4.42 g/L, 0.3 mg/L 6-benzylaminopurine, 1 g/L casein hydrolysate, sucrose preferably at 30 g/L and agar preferably at 7 g/L. pH is preferably adjusted to 5.8. The cultured embryo may be subcultured every 3 to 4 weeks onto either the regeneration or proliferation medium, whichever promotes growth for that interspecific hybrid.

The plantlets that emerge from the cut embryo are a primary plant. As used herein, the term “primary plant” refers to an interspecific hybrid of a Euphorbia plant produced by the method of the first or fourth aspect, and includes the plantlet obtained from culturing the embryo, and plants obtained from incubating cuttings of the plantlet under conditions which result in production of a plant. The primary plant may be propagated by obtaining a cutting and treating the cutting under conditions which stimulate growth of a plant from the cutting. Conditions which stimulate growth of a plant from the cutting may be any conditions that result in production of a plant, and may include treating the cutting, and/or placing the cutting in a particular environment. For example, the cutting may be treated with a composition comprising a hormone capable of inducing root formation. The hormone may be, for example, indole acetic acid (IAA), indole butyric acid (IBA), or napthalene acetic acid (NAA), preferably at 2000 mg/L for a period of between 3 and 20 seconds, preferably 5 seconds. Once the cutting is exposed to the hormone, the cutting is preferably placed in propagation medium. As used herein, a propagation medium is a medium which supports growth of a cutting. An example of a propagation medium may be propagation plugs such as those that are sold under the trademark Jiffy (Jiffy Products). Preferably, a water fog or mist is applied to the plants prior to the development of roots. Primary plants that are planted are preferably planted in suitable potting mix containing commonly used fertilisers known to persons skilled in the art.

To establish whether the primary plant is an interspecific hybrid plant (as opposed to a Euphorbia pulcherrima or Euphorbia species self-pollinated plant), techniques known in the art may be employed such as physical characteristics, genome size, karyotype analysis, hybridisation techniques such as that described by Schwarzacher et al. (1989), In situ localisation of parental genomes in a wide hybrid, Ann. Bot. 64: 315-324, analysis using genotyoping methods such as that described in Starman, T. W., Duan, X. R. and Abbitt, S. (1999) Nucleic acid scanning techniques distinguish closely related cultivars of poinsettia, HortScience 34(6): 1119-1122. For example, interspecific hybrids may be genotyped to determine a representative sample of the inherited markers it possesses relative to the parent plants. Genetic markers are alleles at a single locus. They are preferably inherited in co-dominant fashion so that the presence of both alleles at a diploid locus is readily detectable, and they are free of environmental variation i.e. their heritability is 1. The array of single locus genotypes is expressed as a profile of marker alleles, two at each locus. The marker allele composition of each locus can be either homozygous or heterozygous. Homozygosity is a condition where both alleles at a locus are characterised by the same nucleotide sequence or size or a repeated sequence. Heterozygosity refers to different conditions of the gene at a locus. A preferred type of genetic marker could be used, for example, restriction length polymorphisms (RFLP's), amplified fragment length polymorphism (AFLP's), single nucleotide polymorphisms (SNP's), isozymes, etc. to identify the interspecific hybrid produced by the method of the invention.

It will be appreciated by those skilled in the art that once a primary plant is produced, it may be propagated for an unlimited number of generations. The primary plant may be used to produce further plants with characteristics that are different to that of the primary plant by performing interspecific or intraspecific hybridisation, by grafting the primary plant with other species or cultivars of Euphorbia, or by mutating the primary plant or plants produced from the primary plant.

For example, the primary plant may be used to produce a plant having free-branching characteristics. As used herein, the expression “free-branching characteristics” refers to a characteristic in which lateral branching occurs at a higher frequency than that of a plant that is not free-branching. In order to generate a plant having free-branching characteristics, a free-branching agent is transmitted to the primary plant.

In one embodiment, a free-branching agent may be transmitted to the primary plant by grafting a plant having a free branching agent with the primary plant. The graft used may be any graft which results in transmission of the free-branching agent from the free-branching plant to the primary plant. Preferably, the grafting method used to transmit the agent is an approach grafting method. An approach grafting method involves cutting a section of stem, preferably approximately 10 to 30 mm long and sufficiently deep to reach the cambium, in both the primary plant and the free-branching plant, and subsequently maintaining the cut portions in contact with each other until transfer of the free-branching agent from the free-branching plant to the primary plant has occurred. Cuttings may then be planted and plants having free-branching characteristics grown from the cuttings. In another embodiment, the free-branching agent may be transmitted to the primary plant using a parasitic plant dodder (e.g. Cuscuta sp.). For example, the parasitic dodder may be used to transfer the free-branching agent from a free-branching plant to a non-free-branching plant. Suitably, the parasitic dodder may have the free-branching agent. The use of parasitic dodders for transmitting agents are known in the art and are described in, for example, Lee, I-M., Klopmeyer, M., Bartoszyk, I., Gunderson-Rindal, D., Chou, T., Thomson, K. and Eisenreich, R. (1997), Phytoplasma induced free-branching in commercial poinsettia cultivars, Nature Biotechnology 15: 178-182. In yet another embodiment, the free-branching agent may be transmitted through a leaf hopper. Use of leaf hoppers for transmission of agents between plants is known, and described in, for example, McCoy, R., Caudwell, A., Chang, C., Chen, T., Chiykowski, L., Cousin, M., Dale, J., de Leeuw, G., Golino, D., Hackett, K., Kirkpatrick, B., Marwitz, R., Petzold, H., Sinha, R., Sugiura, M., Whitcomb, R., Yang, I., Zhu, B., Seemuller, E. (1989), Plant diseases associated with mycoplasma-like organisms, In: The Mycoplasmas 5: 545-563.

Examples of free-branching agents capable of being transmitted to the primary plant include phytoplasmas such as poinsettia branch-inducing phytoplasma (PoiBI), or virus such as poinsettia mosaic virus (PnMV) or poinsettia cryptic virus(PnCV). Poinsettia plants carrying one or more of these agents include, for example, cv. V10 Amy red. However, it will be appreciated by those skilled in the art that the free-branching agent may be transferred to practically any Euphorbia pulcherrima of choice and that infected Euphorbia pulcherrima of choice may then be used to transmit the free-branching agent to the primary plant. Confirmation that the free-branching agent has been transmitted to the Euphorbia pulcherrima of choice or the primary plant may be achieved by techniques known in the art such as, for example, morphological examination to note the free branching characteristics of the plant. In addition or alternatively, the actual free-branching agent may be detected using well known techniques such as PCR (polymerase chain reaction), ISEM (immunosorbent electron microscopy), etc.

Examples of free-branching Euphorbia plants that are suitable for use in the method include, for example, the Euphorbia pulcherrima varieties Freedom (U.S. Plant Pat. No. 7,825), Success Red (U.S. Plant Pat. No. 8,773), Red Velvet (U.S. Plant Pat. No. 11,124), Peterstar (U.S. Plant Pat. No. 8,259), Annette Hegg Dark Red (U.S. Plant Pat. No. 3,160), and V-14 Glory (U.S. Plant Pat. No. 4,384).

The characteristics of the plant produced by the method of the invention may be further altered by exposing the plant or a part thereof to a mutagen. The plant or part thereof that is exposed to the mutagen may be any part of the plant from which a mutated plant can be generated. For example, the plant or part of a plant may be the entire plant or a cutting, a shoot, a seed, an embryo, an ovule, a bract, a leaf or any other part of the plant from which a mutated plant can be generated. As used herein, the term “mutagen” refers to a compound or process that results in the introduction of mutations in the plant genome. Examples of suitable mutagens include biological mutagens such as transposons, chemical mutagens such as N-nitroso-N-ethylurea, N-nitroso-N-methylurea, ethylmethane sulfonate (EMS), sodium azide, radiation, or other mutagens. The radiation may be ultra-violet radiation, X-ray radiation, gamma radiation, alpha-radiation, beta-radiation, ion beams such as ⁴He²⁺ and H⁺, etc. Preferably, the radiation is gamma radiation. The dose of gamma radiation will vary depending on the interspecific hybrid, the size of the plant or part thereof and the robustness of the interspecific hybrid. Preferably, the dose of gamma radiation is between 1 and 10,000 rads of gamma radiation. Preferably, the dose is between 1000 and 10000 rads. More preferably, the dose is between 2,000 and 8,000 rads. It is also envisaged that other methods of mutagenesis such as temperature fluctuation, somoclonal variation and mutagen selection through plant tissue culture may be employed to generate plants with different characteristics.

Following mutagenesis, the plant or parts thereof, preferably shoots, are propagated under protocols well known in the art for poinsettia propagation and are described in, for example, (Ecke et al. 1990, The Poinsettia Manual, Paul Ecke Poinsettias, Encinatis, Calif.). Preferably, the apical growth point of the growing plant is routinely removed to encourage branching, and further shoots may be removed to encourage branch formation.

The primary or mutated plant may be propagated by culturing a bract of the primary or mutated plant, by cutting or flowering shoot propagation as described above, or by any other means suitable for propagation of the primary or mutated plant.

In one embodiment, the primary or mutated plant is propagated by culturing a bract of the primary or mutated plant. A bract may be selected and the bract is preferably placed in a solution capable of disinfesting the bract. As used herein, the expression “solution capable of disinfesting” refers to a solution that is able to remove or kill at least a portion of organisms that are located on, or associated with, the bract. Preferably, the solution capable of disinfesting the bract is capable of disinfecting the bract. Preferably, the solution capable of disinfesting the bract is capable of sterilising the bract. Preferably, the solution capable of disinfesting the bract is bleach. Suitably, the bleach solution is at a concentration of between 1% and 3%. Preferably, the bleach solution is at a concentration of about 2%. Preferably, the bract is sterilised by the bleach solution. Accordingly, the bract is preferably placed in the bleach solution for sufficient time to sterilise the bract. Following treatment with the bleach solution, the bleach is preferably replaced with sterile water to thereby wash the bract. Preferably, multiple sterile water washes are carried out. Once the bract is washed, the bract is preferably dissected and placed in contact with culture medium to promote growth. The culture medium may be any culture medium that is sufficient to support growth of a plant from the bract. For example, adventitious root medium as described in (Roest, S. and Bokelman, G. S. (1980). [Vegetative propagation of poinsettias in test-tubes), Vegetatieve vermeerdering van poinsettia in kweenbuizen, Vakblad voor de Bloemisterij 35(47): 36-37, (As translated in: Horticultural Abstracts (1981), 51(6): 430).

Embodiments of the invention are now described in the following Examples which will be understood to merely exemplify and not to limit the scope of the invention.

### EXAMPLES

### Example 1

### Interspecific Hybridisation between Euphorbia pulcherrima and Euphorbia sp.

In this experiment, pollen from Euphorbia cornastra, Euphorbia radians, Euphorbia colorata and Euphorbia fulgens was used to pollinate various cultivars of Euphorbia pulcherrima.

Parental germplasm of Euphorbia pulcherrima used in this experiment are detailed in Table 1.

TABLE 1
Euphorbia pulcherrima parental germplasm used in
experiment

| Euphorbia | |
|---|---|
| pulcherrima | |
| cultivar | Pedigree |
| E. pulcherrima | cv. Freedom white x cv. V10 |
| 97/24.1 | Amy red |
| E. pulcherrima | cv. V10 Amy red x self |
| 97/54.1 | |
| E. pulcherrima | cv. Pink peppermint x cv. V10 |
| 97/144 | Amy red |
| E. pulcherrima | Self seed from wild poinsettia |
| 97/176.3 | |
| E. pulcherrima | Seedling of unknown parentage |
| cv. V10 Amy red | |
| E. pulcherrima | cv. Pepride x [wild-type |
| breeding line 41 | poinsettia x self] |
| E. pulcherrima | cv. Freedom Red x cv. V10 Amy |
| breeding line 75 | Red |
| E. pulcherrima | cv. [Freedom Marble x cv. |
| breeding line 82 | Freedom Red] x [wild-type |
| | poinsettia x self] |

Parental germplasm of Euphorbia cornastra, Euphorbia radians, Euphorbia colorata and Euphorbia fulgens are shown in Table 2.

TABLE 2
Euphorbia species germplasm (other than
pulcherrima) used in experiment

| Euphorbia species | Initial Propagules | Source |
|---|---|---|
| Euphorbia | Seeds | A. Le Duc, |
| cornastra | | Louisiana State |
| | | University, U.S.A. |
| Euphorbia radians | Tubers | V. Steinmann, |
| | | Santa Ana Botanic |
| | | Garden, California |
| | | U.S.A. |
| Euphorbia colorata | Tubers | V. Steinmann, |
| | | Santa Ana Botanic |
| | | Garden, California |
| | | U.S.A. |
| Euphorbia fulgens | Cutting | Readily |
| | | commercially |
| | | available from The |
| | | Plant Place, |
| | | Gosford |

Growth Conditions

Plants used for pollinations were grown in two insect-free environments, namely E1 and E2.

Environment 1 (E1) was a standard environment used for 15 intraspecific-hybridisation. Temperature was maintained at 21° C.±1° C., and a 10 hour photoperiod was provided in “microclimate” rooms contained within a greenhouse. Supplementary light of approximately 300 μmol m⁻² s⁻¹ was provided. The species E. cornastra and the E. pulcherrima cultivars/lines 97/144 and 97/54.1 were grown in environment E1.

In Environment 2 (E2), temperature was maintained between 21° C. and 24° C., natural daylight was provided, and plants were positioned in a northerly aspect to ensure good light intensity. The species E. radians and the poinsettia lines 97/24.1, 97/176.3, and cv. Freedom red, and cv. V10 Amy red were grown in E2 to facilitate crossing with Euphorbia species and also in E1 for all other crosses.

Crossings

Pollinations (self and cross) were performed depending upon flowering times and rate of production of anthers and pistils. Fresh pollen was collected prior to midday from most plants. Pollen was applied liberally to all areas of the receptive stigmatic surface.

Embryo Cutting and Culturing

Cyathia with swollen ovaries were deemed to possess ovules containing fertilised egg cells and were collected prior to abortion. This stage was reached when an abscission layer formed on the cyathium pedicel. Ovaries were disinfected for 10 minutes in 4% NaOCL with 1 drop of Tween 20, then rinsed 3 times in autoclaved distilled water and allowed to dry in a lamina flow cabinet. Upon dissection, all ovules were removed. The ovules were bisected into approximately two halves along their longitudinal axis using a sterile scalpel to ensure that the embryo inside the ovule was cut, and placed cut side upwards onto tissue culture media (regeneration media) containing MS basal salts (Murashige and Skoog 1962), 1 g/l activated charcoal, 1 g/l casein hydrolysate, 40 g/L sucrose and 7 g/l agar. pH was adjusted to 5.8. Media were selected based on previous work (see for example, Roest and Bokelman 1980, Lee et. al. 1997). Primarily plants were cultured at 25° C.±2° C. under Crompton 40W RS White fluorescent lights to provide a light intensity of approximately 60-70 μmol m⁻²s⁻¹ at culture container lid level for 16 hrs/day. Developing embryos were subcultured onto the above mentioned regeneration media or a proliferation media containing MS basal salts, 0.3 mg/l 6-benzyl amino purine, 1 g/l casein hydrolysate, 40 g/L sucrose and 7 g/L agar. pH was adjusted to 5.8. Subsequent subculturing was performed at approximately 3 to 4 week intervals onto fresh media of either composition depending upon growth.

Plantlets (primary plants) developed in vitro were deflasked by either planting the regenerated plantlets emerged directly from embryos, or by cutting and dipping developed shoots in 2000 mg/L IBA for 5 seconds, prior to placement in expanded Jiffy® propagation plugs. A constant water fog was initially applied and later gradually reduced to facilitate acclimatisation once plantlets had developed roots.

Plant Growth

Plants developed in in vitro culture (ovule slice culture) were planted into 150 mm pots containing standard potting mix with supplementary Osmocote® plus 4 month slow release fertiliser applied to the potting mix surface at the recommended rate. Pots were placed at approximately 300 mm interpot distance as measured from the centre of the pots. Water was applied manually and plants were grown under a long photoperiod environment (light intensity greater than 2 μmol m² s⁻¹ for 4 hours commencing at 10 pm) at approximately 25° C. in a greenhouse.

Putative hybrids from the E. pulcherrima×E. cornastra cross (25 hybrids) and two parental controls (cv. V10 Amy red and E. cornastra) were removed from in vitro culture. These plants were acclimatised to the greenhouse environment as described previously and then grown under long photoperiod conditions (light intensity greater than 2 μmol m⁻²s⁻¹ for 4 hours commencing at 10 pm).

Nineteen (19) putative hybrids from the E. pulcherrima×E. cornastra cross (one 97/144×E. cornastra hybrid, one 97/54.1×E. cornastra hybrid) and 17 cv. V10 Amy red×E. cornastra hybrids) and two parental controls (cv. V10 Amy red and E. cornastra) were placed under short photoperiod (10 hours) conditions in environment E1 after approximately 3 months from deflasking. Plants were placed at an interpot distance of 300 mm as measured from the centre of the pots and drip irrigated. Approximately 7 weeks afterwards, bract development in relation to controls was noted for these 19 hybrids.

Infection of E. pulcherrima×E. cornastra Putative Hybrids with PoiBI

Vegetative cuttings were harvested from all stock plants of E. pulcherrima×E. cornastra hybrids and propagated according to standard practices for poinsettias. Cuttings of cv. V10 Amy red containing PoiBI without PnMv were also propagated to enable graft transferral of PoiBI to be conducted. After acclimatisation, each cutting of cv. V10 Amy red was planted in a 150 mm pot adjacent to a cutting of a putative hybrid. Standard potting mix was used, slow release fertiliser was applied at the recommended rate and plants were manually watered and maintained at 25° C.±2° C. under long photoperiod conditions (light intensity greater than 2 μmol m⁻² s⁻² for 4 hours commencing at 10 pm) for several weeks. When the height of both plants was approximately 100 mm, plants were approach grafted.

Approach grafting involved cutting a vertical section of the stem on both plants of approximately 20 to 30 mm in length and deep enough to cut through to the cambium. The two cut portions were then placed facing each other and the graft union sealed with parafilm M laboratory film. Upon development of sufficient growth post-grafting, cuttings were removed from grafted putative hybrids and propagated according to standard protocols. Cuttings with roots were planted into 150 mm pots and grown in a commercial greenhouse according to standard poinsettia production methodology (from vegetative growth to flowering).

Results

E. pulcherrima×Euphorbia sp. Pollinations

A total of 1093 pollinations resulted in 3,279 ovules being pollinated. Unequal pollination numbers resulted among crosses due to different rates of cyathia and anther production. Pollinations between poinsettias and the twelve species yielded at least 1 swollen ovary, from each cross combination, indicating possible fertilisation. Swollen ovaries numbered 243 and contained 689 ovules. All ovules were in vitro cultured only ovules greater than 3 mm in length became organogenic (regenerated structures such as callus, embryos or shoots) when in vitro cultured. Ovules greater than 3 mm in length numbered 370 of which, 119 became organogenic. Ovules 3 mm or less were identical in size and appearance to unpollinated ovule controls and did not exhibit organogenesis in vitro (when harvested just prior to cyathia abortion).

E. pulcherrima 97/24.1×E. radians and cv. V10 Amy Red×E. radians

From a total of 146 crosses conducted among E. pulcherrima and E. radians, 42 swollen ovaries containing 126 ovules resulted. From these ovaries, 36 ovules greater than 3 mm in length were obtained. Of these ovules, 4 exhibited organogenesis.

E. pulcherrima×E. cornastra

Among 380 crosses conducted between poinsettias and E. cornastra, 110 swollen ovaries were observed. From a total of 177 ovules greater than 3 mm in length cultured in vitro, 76 ovules showed organogenesis. The efficiency of production based on the number of plants generated from the number of ovules pollinated ranged from 0 to 27.3%. Higher levels of efficiency were broadly related to fertility of female poinsettia parents. For example, 97/24.1 and 97/54.1 produced 27.3% and 16.7% plants/ovule pollinated respectively.

Crosses which resulted in ovules that exhibited organogenesis in vitro are summarised in table 3.

TABLE 3
Types of in vitro regeneration from interspecific
Euphorbia crosses.

| | | | | Morphological | Plant |
|---|---|---|---|---|---|
| Female | | | Plants | classification | production |
| parent | Male parent | Regeneration* | regenerated | of hybridity | efficiency (%) |
| 97/144 | E. cornastra | 3 (E) | 2 (1 died) | Hybrids | 1.0 (2/198) |
| 97/176.3 | E. cornastra | 3 (E) | 3 | Hybrids | 10.0 (3/30) |
| 97/24.1 | E. cornastra | 10 (E) | 9 (1 died) | Hybrids | 27.3 (9/33) |
| 97/54.1 | E. cornastra | 5 (E) | 5 (1 died) | Hybrids | 16.7 (5/30) |
| | | 1 (G) | | | |
| cv. Angelika | E. cornastra | 1 (E) | 1 | Hybrid | 2.4 (1/42) |
| cv. Freedom | E. cornastra | 1 (E) | 1 | Hybrid | 1.0 (1/102) |
| red | | | | | |
| cv. V10 Amy | E. cornastra | 47 (E) | 44 (3 died) | Hybrids | 12.3 (44/357) |
| red | | | | | |
| 97/24.1 | E. radians.1 | 1 (E) | 1 (1 died) | Hybrid | 1.8 (2/111) |
| | | 2 (G) | | | |
| cv. V10 Amy | E. radians.1 | 1 (C) | 1 | Hybrid | 0.5 (1/183) |
| red | | | | | |
| cv. V10 Amy | E. colorata | 5 (c) | Lost in fire | Hybrid | Destroyed in |
| red | | | shoots | | fire prior to |
| | | | developed | | deflasking |
| cv. 10 Amy | E. fulgens | 2 (c) | Lost in fire | Hybrid | Destroyed in |
| red | | 2 (E) | shoots | | fire prior to |
| | | | developed | | deflasking |
| 97/24.1 | E. fulgens | 1 (c) | Lost in fire | Hybrid | Destroyed in |
| | | 2 (E) | shoots | | fire prior to |
| | | | developed | | deflasking |

*Method of regeneration from ovules: (E) = somatic embryos from zygote; (c) = callus; (G) = direct germination.

Characterisation of Putative F1 Progeny from E. pulcherrima×Euphorbia Species Pollinations
E. pulcherrima×E. cornastra

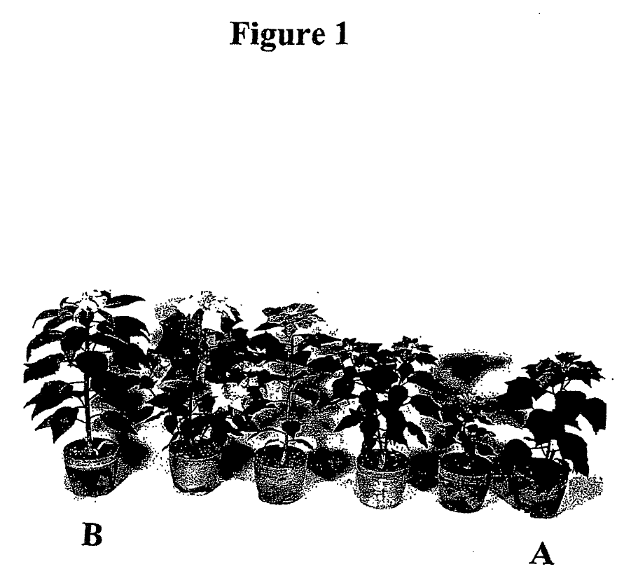

All generated F1 plants displayed characteristics of both poinsettia and E. cornastra parents. In visual comparison to the female poinsettia parents, leaf size was smaller, stem diameter was reduced and internode length was shorter (see FIG. 1 and Table 4). Of the 25 lines characterised for vegetative growth (21 were hybrids between V10 Amy red and E. cornastra), 23 exhibited reduced height (less than E. cornastra) and 15 increased node number, when compared to cv. V10 Amy red (see Table 4). The remaining 4 lines that were not hybrids with cv. V10 Amy red were from crosses with lines 97/144, 97/54.1 and 97/176.3 and were similar in appearance to the other plants.

TABLE 4
Mean Height and node number of interspecific
hybrids.

| | | Means | Hybrid plants |
|---|---|---|---|
| | E. cornastra | and Standard errors | (n = 25, range |
| | (n = 3) | cv. V10 Amy red (n = 5) | of heights) |
| Height (mm) | 522 +/− 51 | 537 +/− 28 | 199-620 |
| Node Number | 26.0 +/− 2.9 | 33.0 +/− 0.71 | 22-47 |
| H/N (mm) | 20.3 +/− 2.0 | 16.3 +/− 0.63 | 7.41-17.3 |

Characterisation of 19 putative hybrids (one 97/144×E. cornastra hybrid, one 97/54.1×E. cornastra hybrid and seventeen cv. V10 Amy red×E. cornastra hybrids) grown under a 10 hour short photoperiod environment showed that 17 exhibited earlier bract development compared to cv. V10 Amy red. The male parent E. cornastra was the earliest to flower, but exhibited rapid bract loss after reaching anthesis. This species also produced numerous seeds, due to self-pollination. Seed production during long photoperiod conditions was not observed for this species, although it did flower under such conditions.

All putative hybrids exhibit pink bract colour with varying degrees of colour intensity. Of the 19 putative hybrid lines observed, 9 exhibited male and female sterility (lack of reproductive structures) and the remaining 10 possessed stamens only.

Considering E. cornastra flowered under long photoperiod conditions, hybrid plants were observed under artificial long photoperiods for 18 months to determine if they would flower. Some lines appeared to commence long photoperiod flowering as indicated by partial colour development of leaves/bracts, but complete development was never observed. When approach grafted to cv. V10 Amy red containing PoiBI, putative hybrids developed swollen buds followed by branches within 2 to 3 weeks of grafting. Cuttings harvested from these free-branching plants possessed increased branching and improved ornamental appearance when grown under commercial conditions indicating transmission of PoiBI. Profuse branching occurred from all nodes regardless of the number of nodes remaining on the primary stem after an apical decapitation (from 6 to 12. nodes). Cuttings produced roots rapidly under standard propagation conditions suitable for poinsettias, and plants grew vigorously.

The characteristics of some examples of the plants that were produced using the method are illustrated in Tables 4 to 8 below.

TABLE 5
Characteristics of an interspecific hybrid
designated 98EC-18.4/5.
98EC-18.4/5

| | |
|---|---|
| Origin: | An interspecific hybrid created by |
| | pollinating a poinsettia plant known as |
| | cultivar V10 Amy Red with pollen from a |
| | Euphorbia cornastra plant developed from |
| | seed |
| Classification: | Euphorbia X hybrid |
| Form: | Soft wooded shrub |
| Infection | Infected with Poinsettia Branch Inducing |
| status: | Phytoplasma (PoiBI) |
| Growth Habit: | Plants were grown according to standard |
| | commercial production procedures for |
| | poinsettias. Finished plant height was |
| | approx. 350 mm and width approx. 550 mm. |
| | The plant had an upright but mounded |
| | habit. The average bract diameter of a |
| | flowering branch was approx. 250 mm. |
| | Branches developing from the main stem |
| | terminate in an inflorescence. If the |
| | main shoot tip is removed prior to |
| | flower induction the plant forms |
| | branches from all nodes present on the |
| | main stem radiating in all directions |
| | due to the removal of apical dominance. |
| | The described plant had 11 nodes |
| | remaining after apical decapitation and |
| | thus 11 branches. Each branch had from |
| | 6-7 leaves. |
| Growth Rate: | Cuttings propagated under mist |
| | (according to standard practices for |
| | poinsettias) produce roots from 10-21 |
| | days. Cuttings can form roots at lower |
| | temperatures than standard poinsettia |
| | cultivars. Plants have medium vigour, |
| | producing one leaf approximately every 5 |
| | days (depending on environmental |
| | conditions). |
| Leaf | Leaves are symmetrical about the mid rib |
| characteristics: | and have a lanceolate appearance. Leaf |
| | length is typically 80-100 mm and width |
| | 30-50 mm. leaf petioles are green with a |
| | slight red tinge on the upper surface |
| | and are typically 20-40 mm long and |
| | approx. 2 mm diameter. The upper leaf |
| | surface is lacking of hairs and slightly |
| | wrinkled, it is mid green in colour (RHS |
| | 137A). Hairs are present on the |
| | underside of leaves and the leaf colour |
| | is lighter green (RHS 137C). Leaf |
| | margins are entire, with some serration |
| | generally closer to the leaf base. |
| Bract | Transitional bracts (bracts with some |
| characteristics: | green and some pink/white colour) |
| | numbered from 1-3 and these are found |
| | between the last leaf produced and the |
| | first bract produced on each branch. |
| | Their shape was lanceolate with some |
| | serration. The upper colour of |
| | transitional bracts was pink with some |
| | green sections. The lower colour was |
| | white with some green sections. Length |
| | was from 70-90 mm and width from 35-45 mm. |
| | The first true bracts produced were from |
| | 80-110 mm in length and 40-60 mm in |
| | diameter. Bract length and width |
| | decreased as they were produced closer |
| | to the cyathia. Bracts directly |
| | subtending the cyathia were from 20-40 mm |
| | long and 10-20 mm wide. Their shape was |
| | symmetrical about the mid rib and |
| | similar to the leaves, but lacking |
| | serration. Upper bract colour was an |
| | intense pink (RHS N57D) and faded as |
| | bracts aged. The underside bract colour |
| | was a light pink/white (close to RHS |
| | 65C). On plants kept for an extended |
| | period of time the upper surface of |
| | older bracts became almost white (close |
| | to RHS 155C). |
| Flower | Plants can be forced to flower by |
| induction: | placing under a ‘short’ photoperiod of |
| | 10 hours. They will naturally flower |
| | under decreasing day length conditions, |
| | similar to a poinsettia. |
| Flowers: | Generally, cyathia numbered from 20-30 |
| | at maturity. Stamens were present on the |
| | more mature cyathia. No pistils were |
| | produced. Mature cyathia were approx. |
| | 8 mm long and 5 mm wide and their colour |
| | was green. |

Colour descriptions are based on RHS colour charts used from 2001 (Royal Horticultural Society, London, 2001)

TABLE 6
Characteristics of an interspecific hybrid
designated 507.1.
507.1

| | |
|---|---|
| Origin: | An interspecific hybrid created by |
| | pollinating poinsettia breeding line 82 |
| | (pedigree: [Freedom Marble X Freedom |
| | Red] X [wild-type poinsettia X self]) |
| | with bulk pollen from several Euphorbia |
| | cornastra plants developed from seed |
| | (via self pollination) |
| Classification: | Euphorbia X hybrid |
| Form: | Soft wooded shrub |
| Infection | Not infected with Poinsettia Branch |
| status: | Inducing Phytoplasma (PoiBI) |
| Growth Habit: | Plants were grown according to standard |
| | commercial production procedures for |
| | poinsettias. Finished plant height was |
| | approx. 450 mm. The plant had an upright |
| | habit. The average bract diameter of a |
| | flowering branch was from 160-180 mm. |
| | Branches developing from the main stem |
| | terminate in an inflorescence. |
| Growth Rate: | Cuttings propagated under mist |
| | (according to standard practices for |
| | poinsettias) produce roots from 10-21 |
| | days. Plants have a comparatively |
| | average rate of growth. |
| Leaf | Leaves are symmetrical about the mid rib |
| characteristics: | and have an oakleaf appearance with 4 |
| | lobes. Leaf length is typically 80-100 mm |
| | and width 65-80 mm. Leaf petioles were |
| | typically 20 mm long and approx. 2 mm |
| | diameter. The upper leaf surface is |
| | green in colour (RHS 139A). The |
| | underside of the leaves is green in |
| | colour (RHS 138A). |
| Bract | Transitional bracts (bracts with some |
| characteristics: | green and some pink colour) numbered |
| | from 2-4 and these were found between |
| | the last leaf produced and the first |
| | bract produced on each branch. Their |
| | shape was oakleaf with four lobes. The |
| | upper colour of transitional bracts was |
| | pink with some green sections. The lower |
| | colour was mostly light green. Length |
| | was from 70-90 mm and width from 50-70 mm. |
| | The first true bracts produced were from |
| | 55-75 mm in length and 35-50 mm in |
| | diameter. Bract length and width |
| | decreased as they were produced closer |
| | to the cyathia. Bracts directly |
| | subtending the cyathia were from 20-30 mm |
| | long and 10-15 mm wide. Their shape was |
| | lanceolate. Upper bract colour was a |
| | dark pink (RHS N66A). The underside |
| | bract colour was pink (RHS N57C). |
| Flower | Plants can be forced to flower by |
| induction: | placing under a ‘short’ photoperiod of |
| | 10 hours. They will naturally flower |
| | under decreasing day length conditions, |
| | similar to a poinsettia. |
| Flowers: | Generally, cyathia numbered from 14-20 |
| | at maturity. Stamens were not present on |
| | mature cyathia. No pistils were |
| | produced. Mature cyathia were approx. |
| | 9 mm long and 6 mm wide and their colour |
| | was green becoming red near the apex. |

Colour descriptions are based on RHS colour charts used from 2001 (Royal Horticultural Society, London, 2001)

TABLE 7
Characteristics of an interspecific hybrid
designated 474.2.
474.2

| | |
|---|---|
| Origin: | An interspecific hybrid created by |
| | pollinating a poinsettia plant known as |
| | poinsettia breeding line 75 (Freedom Red X V10 |
| | Amy Red) with bulk pollen from |
| | several Euphorbia cornastra plants |
| | developed from seed (via self |
| | pollination). |
| Classification: | Euphorbia X hybrid |
| Form: | Soft wooded shrub |
| Infection | Not infected with Poinsettia Branch |
| status: | Inducing Phytoplasma (PoiBI) |
| Growth Habit: | Plants were grown according to standard |
| | commercial production procedures for |
| | poinsettias. Finished plant height was |
| | approx. 300 mm. The plant had an upright |
| | habit. The average bract diameter of a |
| | flowering branch was from 130-140 mm. |
| | Branches developing from the main stem |
| | terminate in an inflorescence. |
| Growth Rate: | Cuttings propagated under mist |
| | (according to standard practices for |
| | poinsettias) produce roots from 10-21 |
| | days. Plants have a comparatively slow |
| | growth rate. |
| Leaf | Leaves are symmetrical about the mid rib |
| characteristics: | and have a semi-oakleaf appearance (2 |
| | lobes). Leaf length is typically 65-80 mm |
| | and width 45-60 mm. Leaf petioles were |
| | typically 15-20 mm long and approx. 2-3 mm |
| | diameter. The upper leaf surface is |
| | green in colour (RHS 137A). The leaf |
| | underside colour is (RHS 137C). |
| Bract | Transitional bracts (bracts with some |
| characteristics: | green and some pink/white colour) |
| | numbered from 3-5 and these are found |
| | between the last leaf produced and the |
| | first bract produced on each branch. |
| | Their shape was semi-oaklef with 2 |
| | lobes. The upper colour of transitional |
| | bracts was light pink with some green |
| | sections. The lower colour was lime |
| | green with some white sections. |
| | Transitional bract length was from 60-70 mm |
| | and width from 35-50 mm. The first |
| | true bracts produced were from 50-60 mm |
| | in length and 20-35 mm in diameter. Bract |
| | length and width decreased as they were |
| | produced closer to the cyathia. Bracts |
| | directly subtending the cyathia were |
| | from 25-35 mm long and 15-20 mm wide. |
| | Their shape was lanceolate. Upper bract |
| | colour was a light pink (RHS 65A). The |
| | underside bract colour was a lighter |
| | pink (RHS 65C). |
| Flower | Plants can be forced to flower by |
| induction: | placing under a ‘short’ photoperiod of |
| | 10 hours. They will naturally flower |
| | under decreasing day length conditions, |
| | similar to a poinsettia. |
| Flowers: | Generally, cyathia numbered from 16-18 |
| | at maturity. Stamens were present on the |
| | more mature cyathia. No pistils were |
| | produced. Mature cyathia were approx. |
| | 7 mm long, 6 mm wide and their colour was |
| | green. |

Colour descriptions are based on RHS colour charts used from 2001 (Royal Horticultural Society, London, 2001)

TABLE 8
Characteristics of an interspecific hybrid
designated 892.1.
892.1

| | |
|---|---|
| Origin: | An interspecific hybrid created by |
| | pollinating a poinsettia plant known as |
| | Poinsettia breeding line 41 (Pedigree: |
| | Pepride X [wild-type poinsettia X self]) |
| | with bulk pollen from several Euphorbia |
| | cornastra plants developed from seed |
| | (via self pollination) |
| Classification: | Euphorbia X hybrid |
| Form: | Soft wooded shrub |
| Infection | Not infected with Poinsettia Branch |
| status: | Inducing Phytoplasma (PoiBI) |
| Growth Habit: | Plants were grown according to standard |
| | commercial production procedures for |
| | poinsettias. Finished plant height was |
| | from 350-400 mm. The plant had an upright |
| | habit. The average bract diameter of a |
| | flowering branch was approx. 90-110 mm. |
| | Branches developing from the main stem |
| | terminate in an inflorescence. |
| Growth Rate: | Cuttings propagated under mist |
| | (according to standard practices for |
| | poinsettias) produce roots from 10-21 |
| | days. Plants have a comparatively |
| | average rate of growth. |
| Leaf | Leaves are symmetrical about the mid rib |
| characteristics: | and have a semi-oakleaf appearance (2 |
| | lobes). Leaf length is typically 90-110 mm |
| | and width 50-60 mm. Leaf petioles |
| | were typically 20-25 mm long and approx. |
| | 2 mm diameter. The upper leaf surface is |
| | green in colour (RHS 139B). The |
| | underside leaf colour is green (RHS |
| | 139C). |
| Bract | Transitional bracts (bracts with some |
| characteristics: | green and some pink/white colour) |
| | numbered from 3-4 and these are found |
| | between the last leaf produced and the |
| | first bract produced on each branch. |
| | Their shape was lanceolate. The upper |
| | colour of transitional bracts was pink |
| | with some green sections. The lower |
| | colour was green with some very pale |
| | pink sections. Transitional bract length |
| | was from 40-60 mm and width from 25-35 mm. |
| | The first true bracts produced were from |
| | 35-45 mm in length and 20-35 mm in width. |
| | Bract length and width decreased as they |
| | were produced closer to the cyathia. |
| | Bracts directly subtending the cyathia |
| | were from 20-25 mm long and 10-20 mm wide. |
| | Their shape was lanceolate. Upper bract |
| | colour was medium pink (RHS N57D). The |
| | underside bract colour was a lighter |
| | pink (RHS 65C). |
| Flower | Plants can be forced to flower by |
| induction: | placing under a ‘short’ photoperiod of |
| | 10 hours. They will naturally flower |
| | under decreasing day length conditions, |
| | similar to a poinsettia. |
| Flowers: | Generally, cyathia numbered from 11-16 |
| | at maturity. Stamens were present on the |
| | more mature cyathia. No pistils were |
| | produced. Mature cyathia were approx. |
| | 8 mm long and 5 mm wide and their colour |
| | was green becoming pink near the apex. |

Colour descriptions are based on RHS colour charts used from 2001 (Royal Horticultural Society, London, 2001)

TABLE 9
Characteristics of an interspecific hybrid
designated 674.3.
674.3

| | |
|---|---|
| Origin: | An interspecific hybrid created by |
| | pollinating a poinsettia plant known as |
| | cultivar V10 Amy Red with pollen from |
| | several Euphorbia cornastra plants |
| | developed from seed (via self |
| | pollination) |
| Classification: | Euphorbia X hybrid |
| Form: | Soft wooded shrub |
| Infection | Not infected with Poinsettia Branch |
| status: | Inducing Phytoplasma (PoiBI) |
| Growth Habit: | Plants were grown according to standard |
| | commercial production procedures for |
| | poinsettias. Finished plant height was |
| | approx. 800-850 mm. The plant had an |
| | upright habit. The average bract |
| | diameter of a flowering branch was 130-150 mm. |
| | Branches developing from the main |
| | stem terminate in an inflorescence. |
| Growth Rate: | Cuttings propagated under mist |
| | (according to standard practices for |
| | poinsettias) produce roots from 10-21 |
| | days. Plants have a comparatively fast |
| | rate of growth. |
| Leaf | Leaves are symmetrical about the mid rib |
| characteristics: | and have a lanceolate appearance. Leaf |
| | length is typically 140-160 mm and width |
| | 60-90 mm. Leaf petioles are typically 40-45 mm |
| | long and approx. 2 mm diameter. The |
| | upper leaf surface is green in colour |
| | (RHS 137B). The underside leaf colour is |
| | lighter green (RHS 137C). |
| Bract | Transitional bracts (bracts with some |
| characteristics: | green and some pink/white colour) |
| | numbered from 4-6 and these are found |
| | between the last leaf produced and the |
| | first bract produced on each branch. |
| | their shape was lanceolate. The upper |
| | colour of transitional bracts was pink |
| | with some green sections. The lower |
| | colour was very pale green with some |
| | white sections. Transitional bract |
| | length was from 80-100 mm and width from |
| | 35-45 mm. The first true bracts produced |
| | were from 45-60 mm in length and 15-25 mm |
| | in diameter. Bract length and width |
| | decreased as they were produced closer |
| | to the cyathia. Bracts directly |
| | subtending the cyathia were from 20-35 mm |
| | long and 10-15 mm wide. Their shape was |
| | symmetrical about the mid rib and |
| | similar to the leaves. Upper bract |
| | colour was a light pink (RHS 63C). The |
| | underside bract colour was a lighter |
| | pink (RHS 65D). |
| Flower | Plants can be forced to flower by |
| induction: | placing under a ‘short’ photoperiod of |
| | 10 hours. They will naturally flower |
| | under decreasing day length conditions, |
| | similar to a poinsettia. |
| Flowers: | Generally, cyathia numbered from 20-65 |
| | at maturity. Stamens were not present on |
| | mature cyathia. No pistils were |
| | produced. Mature cyathia were approx. |
| | 10 mm long and 9 mm wide and their colour |
| | was green. |

Colour descriptions are based on RHS colour charts used from 2001 (Royal Horticultural Society, London, 2001).

It will be understood by those skilled in the art that plant characteristics are variable under different environmental conditions and thus results obtained in the above table 5 to 9 may vary somewhat under different growing conditions.

Conclusions

The use of ovule slice culture to culture the growing embryo has been used to produce crosses between a number of different Euphorbia species.

### Example 2

### Irradiation of Interspecific Hybrids to Produce New Varieties

The aim of this experiment was to determine whether plants could be produced having variation in characteristics from the primary plants by irradiating E. pulcherrima×E. cornastra F₁ hybrids.

Materials and Methods:

One interspecific hybrid (98EC-18.4/5) was selected. The characteristics of this interspecific hybrid are listed in Table 5 above.

Cuttings were obtained from plant 98EC-18.4/5. The cuttings were made to obtain healthy and uniform tissue having a high node number. Unnecessary leaves were removed with a scalpel.

Irradiation of cuttings was performed as shown in Table 10. All cuttings were covered with plastic freezer bags. The Gamma source used emitted irradiation at approximately 500 rads per 10 minutes.

Five experimental treatments for the cuttings (control and T1 to T4) were set up. The details of each of the treatments are shown in Table 10.

TABLE 10
Irradiation treatment of cuttings.

| Treatment type | Dose (Rads) | Treatment Time (minutes) |
|---|---|---|
| C | 0 (control) | 0 (leave in box) |
| T1 | 2000 | 43 |
| T2 | 3000 | 64 |
| T3 | 4000 | 80 |
| T4 | 8000 | 171 |

Cuttings were set up in 6×20 cm diameter vials and rotated at 15 cm from the gamma source. Once treated, cuttings were rewrapped in fresh moistened paper and placed back into polystyrene boxes.

Irradiated (and control) cuttings were placed into Oasis® cubes (Smithers-Oasis) and propagated via standard commercial practice, under mist with bottom heat (22° C.) (Ecke et al. 1990). After 8 weeks all plants with roots were potted following standard practices (Ecke et al. 1990).

Results

Results are summarised in Table 11.

TABLE 11
Irradiation results.

| | | Treatment | No. surviving | | | |
|---|---|---|---|---|---|---|
| Cutting | | time | after | | after 8 | |
| No. | Dose (Rads) | (minutes) | 1 week | % | weeks | % |
| 55 | 0 (control) | 0 | 55 | 100 | 55 | 100 |
| 102 | 2000 (T1) | 43 | 102 | 100 | 96 | 94 |
| 102 | 3000 (T2) | 64 | 102 | 100 | 95 | 93 |
| 102 | 4000 (T3) | 80 | 102 | 100 | 72 | 70 |
| 102 | 8000 (T4) | 171 | 102 | 100 | 5 | 5 |

At 3 months post-irradiation (4 weeks past potting), shoots were apically decapitated to promote branching. All plants except controls had speckled green and light green patches on leaves from radiation.

At 6 months post-irradiation, shoots of all plants were further apically decapitated to promote branching.

After approximately 6 months, all plants were taken to a short photoperiod environment (10 hour day length) to flower.

After 7.5 months, plants were flowering. Selections were made for phenotypic attributes such as bract colour and shape, leaf variegation and plant habit. Subsequently all branches on selected plants except the mutated branches were removed.

After 8.5 months, the best plants were reselected from primary selections.

After 9 months, all remaining stock (unselected) was cutback and allowed to reflower. The first batch of flowering shoot cuttings were taken from selected, mutated branches. Flowering shoots containing mutations were propagated following standard propagation procedures for Poinsettia (Ecke 1990).

Interspecific hybrid mutants were selected and propagated in propagation media under standard conditions for propagation of poinsettia cuttings, and in tissue culture via bract culture (see Example 3) Results are summarised in Table 12 which shows the number of plants with visible, useful mutations.

TABLE 12
Summary of interspecific hybrid mutagenesis.

| | | Treatment | | No. of | % mutant |
|---|---|---|---|---|---|
| Cutting | | Time | No. | mutant | production |
| no. | Dose (Rads) | (minutes) | potted | selections | efficiency |
| 55 | 0 (control) | 0 | 55 | 0 | 0 |
| 102 | 2000 (T1) | 43 | 96 | 51 | 50% |
| 102 | 3000 (T2) | 64 | 95 | 38 | 37% |
| 102 | 4000 (T3) | 80 | 72 | 46 | 45% |
| 102 | 8000 (T4) | 171 | 5 | 11 | 11% |

The resulting mutants produced by the mutagenesis experiment are shown in Table 13.

TABLE 13
Mutants produced by gamma irradiation of
interspecific hybrid Euphorbia 9 months post-irradiation.

| | | | | New | | | |
|---|---|---|---|---|---|---|---|
| | Selected | | | Shoots | | | % bract |
| Treatment | shoot | Cuttings | | in | | RHS | area |
| level | number | taken | Details | culture | Description | colour | (approx) |
| T1. | 1 | 2 | stable | 2 | white, pk veins | 155C | ½ |
| T1. | 2 | 0 | no white | 4 | white | 155C | ½ |
| T1. | 3 | 3 | 2S 1NS | | white | N155D | ½ |
| T1. | 4 | | NS | 1 | white | 69D | ½ |
| T1. | 5 | | | | white | 155C | <¼ |
| T1. | 6 | | NS | 1 | pk, dk pk speckle | 65A | ½ |
| T1. | 7 | | | | pk, dk pk speckle | | ½ |
| T1. | 8 | | | | lt pk | | full |
| T1. | 9 | 1 | NS | 1 | white, pk speckle | | ¾ |
| T1. | 10 | | | | lt pk | | ¾ |
| T1. | 11 | 2 | 1S 1NS | 2 | wh/v lt pk | | ½ |
| T1. | 12 | 0 | nil pink | 2 | lt pk | N155C | full |
| T1. | 13 | 3 | NS | 1 | white, pk speckle | 155C | full |
| T1. | 14 | 1 | NS | 2 | v lt pk | | ½ |
| T1. | 15 | 4 | NS | | white | | ¼ |
| T1. | 16 | 1 | NS | 1 | lt pk | | ½ |
| T1. | 17 | 1 | NS | | white | 155C | ¾ |
| T1. | 18 | 3 | fairly | | lt pk | | full |
| | | | stable | | | | |
| T1. | 19 | 3 | stable | 2 | baby pk | 69C | full |
| T1. | 20 | | | | variegated jagged | | ? |
| | | | | | edge | | |
| T1. | 21 | | | | white, pinkish | | ¼ |
| | | | | | splotches | | |
| T1. | 22 | | | | white | | <¼ |
| T1. | 23 | 4 | fairly | | hot pink | 63B | full |
| | | | stable | | | | |
| T1. | 24 | | | | lt pk | | full |
| T1. | 25 | | | | lt pk | | full |
| T1. | 26 | | | | baby pk | | full? |
| T1. | 27 | 5 | 3stable + 2FS | 2 | white | 155C | full |
| T1. | 28 | | no shoot | 1 | Brightest pk | | ½ |
| | | | | | (blotchy) | | |
| T1. | 29 | 1 | no specs | | Brightest pk | | ¼ |
| | | | | | (speckle) | | |
| T1. | 30 | 3 | NS | | normal pk, | | full |
| | | | | | variegated grey/wh | | |
| | | | | | edge | | |
| T1. | 31 | 2 | NS | | normal pk, wh | | full? |
| | | | | | patches, | | |
| | | | | | variegated | | |
| T1. | 32 | 3 | 1SW 1SP | 2 | wh/bright pk | | ⅛ |
| | | | 1both | | | | |
| T1. | 33 | 4 | NS | | normal pk, | | ¼ |
| | | | | | variegated, wh/pk | | |
| | | | | | edge | | |
| T1. | 34 | 1 | S | | pk, w bright pk | | ¾ |
| | | | | | blotches | | |
| T1. | 35 | | | | white? | | |
| T1. | 36 | | | | normal | | |
| T1. | 37 | | | | normal | | |
| T1. | 38 | 6 | S | 1 | pk, jingle bells, | 62B | full |
| | | | | | dk and lt pk | | |
| | | | | | blotches | | |
| T1. | 39 | 6 | 3Slight | | mid pk(2shades 1 | 63B | ½ |
| | | | 3Sdark | | slightly lighter) | | |
| T1. | 40 | 1 | S | | pale pink | 69D | full |
| T1. | 41 | 1 | S | | bright pink | | ¾ |
| T1. | 42 | 1 | S | | bright pink | | full |
| T1. | 43 | 1 | S | | very pale | N155D | full |
| | | | | | pink/cream | | |
| T1. | 44 | 1 | S | | bright pink | N66A | full |
| T1. | 45 | 1 | S | | bright pink | | full |
| | | | | | cyathia | | |
| T1. | 46 | 1 | NS | | white with few | | full |
| | | | | | bright pink flecks | | |
| T1. | 47 | 1 | S | | pale pink speckle | | ¾ |
| T1. | 48 | 1 | NS | | white with pink | | full |
| | | | | | speckle mixed | | |
| T1. | 49 | 1 | NS | | jingle bell white | | ¾ |
| | | | | | speckles | | |
| T1. | 50 | 1 | S | | pale pink | | full |
| T1. | 51 | 1 | S | | bright pink white | 62A | full |
| | | | | | flecks pink | | |
| | | | | | cyathia | | |
| T2. | 1 | 4 | stable | 4 | pk w wh patches | | full |
| | | | | | (jingle bells | | |
| | | | | | type) | | |
| T2. | 2 | 4 | | | pk, variegated | | ½ |
| | | | | | grey | | |
| T2. | 3 | 1 | | 1 | brightest pk | N66A | ¼ |
| T2. | 4 | 1 | | 1 | pk/wh patches | | ½ |
| T2. | 5 | 1 | 2stable | 3 | baby/v lt pk | N155C | ½ |
| | | | 1NS | | | | |
| T2. | 6 | | | | normal? | | |
| T2. | 7 | 2 | S | 1 | Brightest pk | | ¼ |
| T2. | 8 | 0 | missing | | wh, sl pk | | ⅓ |
| T2. | 9 | | | | normal | | |
| T2. | 10 | | | | white | | <⅛ |
| T2. | 11 | 4 | | | normal pk, | | full |
| | | | | | variegated, wh/pk | | |
| | | | | | jagged edge | | |
| T2. | 12 | 0 | tiny | 1 | wh/baby pk | 69D/ | ¼ |
| | | | sector | | | 155C | |
| T2. | 13 | | | | wh | | <⅛ |
| T2. | 14 | 3 | | | normal pk, | | ¾ |
| | | | | | variegated, wh/pk | | |
| | | | | | jagged edge | | |
| T2. | 15 | 4 | | | normal pk, | | full |
| | | | | | variegated, wh/pk | | |
| | | | | | jagged edge | | |
| T2. | 16 | 2 | NS | | baby pk | 69C | full |
| T2. | 17 | 3 | S | 1 | hot pk | 63B | full |
| T2. | 18 | 2 | 1S + 1NS | 1 | baby pk | | ½ |
| T2. | 19 | 2 | S | | white | | full |
| T2. | 20 | 3 | S | | hot pk | | ½ |
| T2. | 21 | 5 | S | | baby pk, brighter | 69C | full |
| | | | | | new bracts | | |
| T2. | 22 | 3 | no change | 4 | lt pk | | full |
| T2. | 23 | 2 | S | 1 | wh, v lt pk | | ½ |
| T2. | 24 | 3 | S | 2 | lt/normal pk, wavy | | full |
| | | | | | bracts | | |
| T2. | 25 | 4 | S | 1 | v lt pk? | | ¾ |
| T2. | 26 | 2 | 1S light | 1 + 1 | hot pk and white | 63A | ½ |
| | | | 1S dark | | | | |
| T2. | 27 | 1 | S | | mid pk | 63B | full |
| T2. | 28 | 1 | S | | pale pink | 69B | full |
| T2. | 29 | 1 | S | | pale pink | 69B | full |
| T2. | 30 | 1 | NS | | pale pink with | 62B | full |
| | | | | | speckles | | |
| T2. | 31 | 1 | NS | | very bright pink | | ¼ |
| | | | | | sector | | |
| T2. | 32 | | | | | | |
| T2. | 33 | 1 | S | | pale pink | | full |
| T2. | 34 | 1 | S | | white | | full |
| T2. | 35 | 1 | S | | pale pink | | full |
| T2. | 36 | 1 | S | | bright pink white | 68A | full |
| | | | | | speckle | | |
| T2. | 37 | 1 | S | | white/very pale | | full |
| | | | | | pink | | |
| T2. | 38 | 1 | S | | pale pink/pink | | full |
| | | | | | veins | | |
| T3. | 1 | 1 | S | | wh, pk veins | 155C | ½ |
| T3. | 2 | 2 | S | | lt pk, variegated | | ¾ |
| | | | | | wh edges | | |
| T3. | 3 | 2 | S | | pk, variegated wh | 63C | full |
| | | | | | edges pk/wh on | | |
| | | | | | trans bracts | | |
| T3. | 4 | | | | wh | | ¼ |
| T3. | 5 | 2 | 1S 1NS | 1 | wh, v lt pk | | ½ |
| T3. | 6 | | | | pk, variegated, | 67D | full |
| | | | | | wh/lt green edges, | | |
| | | | | | distorted | | |
| T3. | 7 | 0 | missing | | pk, wh edged | 63C | ½ |
| | | | | | variegation | | |
| T3. | 8 | 0 | missing | 2 | pk w wh blotches | 63C | ¾ |
| T3. | 9 | 4 | S | | pk, variegated | | ⅞ |
| | | | | | grey, jagged edge | | |
| T3. | 10 | 4 | S | 1 | baby pk | 64B | ½ |
| T3. | 11 | | | | ugly variegated pk | | full |
| T3. | 12 | 2 | 1S 1NS | | wh | | ½ |
| T3. | 13 | 2 | NS | 1 | wh | | ½ |
| T3. | 14 | 3 | NS | | pk/lt pk blotches | | full |
| T3. | 15 | 0 | broken | | lt baby pk | | ½ |
| | | | off | | | | |
| T3. | 16 | 4 | NS | | pk w dk pk speckle | | full? |
| T3. | 17 | 0 | missing | 2 | wk, pk new bracts | | full |
| T3. | 18 | 0 | missing | | pk, grey | | ½ |
| | | | | | variegated | | |
| T3. | 19 | 4 | NS | | pk, grey | 63C | full |
| | | | | | variegated | | |
| T3. | 20 | 3 | 1S baby | 2 + 1 | baby pk and v lt | | ½ and |
| | | | 1Slight | | pk | | ½ |
| | | | 1NS | | | | |
| T3. | 21 | 4 | 1light | | pk variegated w | 69A | full |
| | | | | | wh edge, jagged | | |
| T3. | 22 | 2 | S | | hot pk | | full |
| T3. | 23 | 5 | S | | pk, variegated, wh | 63C | full |
| | | | | | edge | | |
| T3. | 24 | 1 | NS | | mid pink | | ½ |
| T3. | 25 | | | | baby pk | | ⅓ |
| T3. | 26 | 2 | S | 1 | hot pk | N66A | ½ |
| T3. | 27 | 3 | S | | baby pk and hot pk | | ¼ |
| T3. | 28 | 0 | nil | 1 | hot pk | | full |
| | | | cuttings | | | | |
| T3. | 29 | 1 | S | | v lt pk | | ¾ |
| T3. | 30 | | | | wh | | <¼ |
| T3. | 31 | 1 | S | | wh | | full |
| T3. | 32 | 2 | no change | | lt pk | | full |
| T3. | 33 | 2 | S | | pk, variegated | 65A | ? |
| T3. | 34 | | | | small variegated | | ⅓ |
| | | | | | leaf, wh edge | | |
| T3. | 35 | 3 | S | | pk, wh speckle on | | full |
| | | | | | leaves like LD | | |
| | | | | | initn | | |
| T3. | 36 | 1 | S | 2 | bright pk | | ⅓ |
| T3. | 37 | 3 | S | 2 | baby pk, | 65D | full, |
| | | | | | variegated | | ½ |
| | | | | | | | varieg |
| T3. | 38 | 4 | S | | pale pk/wh | 62D | full |
| T3. | 39 | 1 | S | | white | | full |
| T3. | 40 | 2 | NS | | bright pk with | | ½ |
| | | | | | speckles serrated | | |
| | | | | | bracts | | |
| T3. | 41 | 1 | S | | white | | full |
| T3. | 42 | 2 | S | | pale pink | 69B | full |
| T3. | 43 | 1 | NS | | bright pink with | | ¼ |
| | | | | | speckles | | |
| T3. | 44 | 1 | NS | | pale pink | | full |
| T3. | 45 | 1 | S | | white | | full |
| T3. | 46 | 2 | NS | | jingle bells brt | 63B | |
| | | | | | pk | | |
| T4. | 1 | 0 | broken | | jingle bells type | | full |
| T4. | 2 | 3 | NS | 1 | medium pk | N66A | ½ |
| T4. | 3 | 5 | S | | pk w bright | | ½ |
| | | | | | patches | | |
| T4. | 4 | 4 | 1S 3NS | 1 | baby pk | | full |
| T4. | 5 | 0 | | | baby pk, | | full, |
| | | | | | variegated, bit | | ½ |
| | | | | | distorted | | varieg |
| T4. | 6 | 3 | S | | pk, variegated, | | ½ |
| | | | | | distorted | | |
| T4. | 7 | 2 | S | | pk, small leaf | | full |
| | | | | | variegated w wh | | |
| | | | | | edge | | |
| T4. | 8 | 1 | S | | lt pk and baby pk | 62A | ½ and |
| | | | | | | | ½ |
| T4. | 9 | 3 | S | 1 | wh, distorted fol | | full |
| | | | | | and bracts | | |
| T4. | 10 | | | | pk, wh speckle on | | ¼ |
| | | | | | leaves like LD | | |
| | | | | | initn | | |
| T4. | 11 | 1 | S | | white with cream | | ¾ |
| | | | | | yellow tinge | | |

*Colours include, standard or normal pink (same as control), brightest pink (br pk), hot pink (hot pk), light pink (lt pk), baby pink (baby pk), white (wh), white jingle bells (white with pink fleck), pink jingle bells (pink with white fleck), pink centre (white with small new bracts pink). No tissue culture except where stated.
v = very.
w = with.
NS = not stable,
S = stable,
FS = fairly stable

Conclusion

Irradiation of interspecific hybrids produced from a cross between Euphorbia pulcherrima and Euphorbia cornastra resulted in a variety of plants with altered characteristics including colour and variegation.

### Example 3

### Stabilisation of Chimeric Mutants

Once a desirable mutation was observed, a cutting was taken of the flowering shoot. It will be appreciated by those skilled in the art that the greater the mutated section of the shoot, the greater the chance of stabilising the phenotype in the next cutting generation. It is desirable for the cutting to have most of its bracts removed and to possess at least two green leaves. The cuttings can be propagated following standard procedures suitable for poinsettias. The cuttings are then grown to produce flowering plants following standard procedures for poinsettias. At this stage, shoots that have the desirable mutant phenotype are selected and propagated. The cycle continues until the desirable mutant is stable (non-chimeric).

### Example 4

### Bract Tissue Culture Experiment for Interepecific Euphorbia Hybrid

Many of the plants produced from the irradiation experiment had sections or portions of the plant which exhibited desirable characteristics. In other words, the plants were chimeric. The differences in the characteristics of these portions were presumably due to somatic mutations resulting from the irradiation. This example describes the development of a tissue culture system in which the mutant portions can be cultured to produce non-chimeric shoots from desirable mutated sections of a chimeric plant, such as those with novel colours arising from gamma irradiation treatment.

Eighteen bracts and leaves of medium size and age were collected with petioles intact from the interspecific hybrid designated 98EC18.4/5. The following concentrations of bleach (NaOCl Zixo brand 4.5% active) were prepared:
1% bleach
2% bleach
3% bleach

The bracts and leaves were divided into 6 treatment groups listed in table 14 and 15. A separate conical flask was used for each treatment/time combination (=6 for bracts and 6 for leaves).

TABLE 14
Treatment groups for bract culturing.

Treatment group

| | | | | | |
|---|---|---|---|---|---|
| 1 | 2 | 3 | 4 | 5 | 6 |

| | | | |
|---|---|---|---|
| Bleach (%) | 1% | 2% | 3% |

| | | | | | | |
|---|---|---|---|---|---|---|
| Time (min) | 5 | 10 | 5 | 10 | 5 | 10 |
| Bract no. | 3 | 3 | 3 | 3 | 3 | 3 |

TABLE 15
Treatment groups for leaf culturing.

Treatment group

| | | | | | |
|---|---|---|---|---|---|
| 1 | 2 | 3 | 4 | 5 | 6 |

| | | | |
|---|---|---|---|
| Bleach (%) | 1% | 2% | 3% |

| | | | | | | |
|---|---|---|---|---|---|---|
| Time (min) | 5 | 10 | 5 | 10 | 5 | 10 |
| Leaf no. | 3 | 3 | 3 | 3 | 3 | 3 |

Bracts and leaves were placed into labelled separate flasks. One drop of tween 20 was then added to each flask and approximately 50 mL of the appropriate concentration of bleach was added. The flasks were then sealed with a stopper (ensuring that plant material was covered with the bleach solution) and shaken vigorously to ensure all areas inside the flask were covered in bleach solution. The plant material was allowed to settle, and was re-shaken about once or twice per minute. Following the required time, the bleach was poured off and 200 mL of autoclaved water was added to the plant material in the flask. The flask was then shaken once per minute for 5 minutes. The water was subsequently poured off and a further 200 ml added. This was repeated twice for a total of three rinses of 5 min each. After the last rinse with water, more water was added and the material was allowed to stay in the flasks until required for culturing.

Following bleach treatment, bleach affected (white) areas of the bracts or leaves were removed. The remaining bracts and leaves were then dissected into irregular sized fragments of 1-2 cm diameter and placed into jars containing adventitious shoot media (ASM) (Roest and Bokelman, 1980) containing 4.42g/L MS salts, 3% sucrose, 0.7% agar, pH 5.8, 2g/L Myo-inositol, 1.0 mg/L 6-Benzylaminopurine (BAP), 0.1 mg/L 1-Napthaleneacetic acid (NAA). Five fragments were placed in each jar and the fragments incubated at 25° C.±2° C. under 40W white fluorescent lights providing an intensity of 60-70 μmol m⁻²s⁻¹ at culture container lid level for 16 h/day.

Results

Results after 4.5 weeks indicated a bleach treatment of 1% for 10 min to 2% for 10 min produced the least contaminated cultures with the least amount of over-bleaching.

Callus was produced from bracts. Callus was produced from the mutated interspecific hybrid leaves, but its growth was much less than other explants. Where callus was produced this was evident from 2-3 weeks after initiation. Interspecific hybrid bracts turned from pink to a greenish colour and initiated callus in particular where they contacted the media.

All of the surviving bracts produced shoots on ASM medium after about 4 weeks. Shoots arose from the callus. Shooting clumps were then cut and planted onto multiplication medium to multiply for several weeks and then shoots were cut and planted into a greenhouse propagation facility following standard procedures used for poinsettias.

TABLE 16
Results of leaf and bract culture of Euphorbia
interspecific hybrid 98EC18.4/5 3.5 and 4.5 weeks after in
vitro initiation

| | | Results (3.5 wks) | Results (4.5 wks) |
|---|---|---|---|
| | Treatment | (3 jars per | (3 jars per |
| Explant | (see Tables | treatment, 5 | treatment, 5 |
| type | 14 and 15) | explants per jar) | explants per jar) |
| leaves | 1 | very few with | dead explants |
| | | callus, nil dead | recorded: 0/5, |
| | | | 0/5, 2/5 |
| leaves | 2 | few with callus | 0/5, 0/5, 2/5 dead |
| | | on edge, nil dead | |
| leaves | 3 | more callus on | some damage |
| | | edge, some leaf | |
| | | damage | |
| leaves | 4 | no callus, some | 4/5, 2/5, 2/5 dead |
| | | leaf damage | |
| leaves | 5 | some callus on | 1/5, 3/5, 2/5 dead |
| | | edge, especially | |
| | | near vein, some | |
| | | leaf damage | |
| leaves | 6 | 1 dead. Heavy | 4/5, 4/5, 4/5 dead |
| | | leaf damage, no | |
| | | callus | |
| bracts | 1 | all contaminated | all contaminated |
| bracts | 2 | all contaminated | all contaminated |
| bracts | 3 | 1 jar remains, 3 | 3 callus + shoots |
| | | green/red callus | 2/5 dead explants |
| | | edges | |
| bracts | 4 | 1 jar remains, 3 | 3 callus + shoots |
| | | green/red callus | 2/5 dead explants |
| | | edges | |
| bracts | 5 | 1 jar remains, 1 | 1 callus + shoots |
| | | piece green | 3/5 dead |
| | | callus edges | |
| bracts | 6 | all contaminated | all contaminated |

Conclusion:

The use of 2% bleach for 10 minutes for treating bracts is recommended for disinfestation resulting in optimal success for explant introduction to culture.

Shoots can be produced from callus derived from interspecific hybrid bracts after several weeks in culture growing on ASM media.

Following this experiment bracts from mutated interspecific hybrid selections (Table 13) were introduced into culture following the protocols described above, and plants were regenerated. The plants were grown in a greenhouse and then placed under floral inducing conditions (10 hr photoperiod) to allow them to flower. Upon flowering it was observed that one line [treatment T2, shoot number 5 from Table 13 (mutant T2.5)] which was labelled very light pink had become non-chimeric (stable) and retained the very light pink colour. The mutant non-chimeric plants obtained from bract culture of mutant T2.5 appeared phenotypically very similar to the primary plant 98EC18.4/5 (RHS colour N57D, pink), except that bract colour was close to RHS N155C, very light pink to almost white. Several plants grown of this line were all non-chimeric.

1. A method for producing an interspecific hybrid Euphorbia plant comprising:
(a) providing a first plant which is a Euphorbia pulcherrima plant and a second plant which is a species of Euphorbia selected from the group consisting of Euphorbia cornastra, Euphorbia radians, Euphorbia colorata and Euphorbia fulgens;
(b) pollinating a flower of the second plant with pollen from the first plant or a flower of the first plant with pollen from the second plant in a manner which permits formation of an embryo in at least one ovule of the pollinated plant;
(c) cutting the embryo; and
(d) culturing the cut embryo by placing the cut embryo in contact with culture medium to permit growth of the embryo to thereby produce a primary plant.

2. The method of claim 1 wherein a flower of the first plant is pollinated with pollen from the second plant.

3. The method of claim 2 wherein the second plant is selected from the group consisting of Euphorbia cornastra, Euphorbia radians, Euphorbia colorata and Euphorbia fulgens.

4. The method of claim 1 wherein the embryo is cut while contained in the ovule.

5. The method of claim 1 wherein the ovule is at least 3 millimetres in length.

6. The method of claim 1 wherein the ovule is sliced transverse to the longitudinal axis, or along the longitudinal axis.

7. The method of claim 6 wherein the ovule is sliced along the longitudinal axis of the ovule.

8. The method of claim 1 wherein the culturing step employs ovule slice culture.

9. The method of claim 1 wherein the embryo is cut following excision from the ovule.

10. The method of claim 1 wherein the embryo is cut following embryo rescue.

11. The method of claim 1 wherein the embryo is excised from an ovule that is at least 3 millimetres in length.

12. The method of claim 1 further comprising the steps of:
(a) obtaining a cutting from the primary plant;
(b) incubating the cutting under conditions sufficient to propagate the primary plant.

13. The method of claim 1 wherein the cutting is a shoot.

14. The method of claim 1 wherein the cutting is treated to induce root formation.

15. The method of claim 14 wherein the root formation is induced by treating the cutting with a composition containing a hormone capable of inducing root formation.

16. The method of claim 15 wherein the hormone is an auxin.

17. The method of claim 1 comprising the further step of transmitting a free-branching agent to the primary plant.

18. The method of claim 1 wherein the free branching agent is transmitted to the primary plant by a dodder (a parasitic plant), by a leaf hopper insect or by a graft.

19. The method of claim 1 wherein the free-branching agent is transmitted to the primary plant by:
(a) providing a free-branching plant having a free-branching agent;
(b) cutting the primary plant and the free-branching plant to expose tissue of the plant;
(c) making a graft union between the tissue of the free-branching plant and the primary plant, whereby at least one characteristic of a vegetative shoot arising from said graft is different from the free-branching plant and the primary plant; and
(d) growing said shoot to obtain a grafted primary plant with at least one altered growth characteristic.

20. The method of claim 19 wherein the free-branching agent is a phytoplasma.

21. The method of claim 1 further comprising the steps of:
(a) obtaining a cutting of the primary plant or the grafted primary plant;
(b) exposing the cutting to a mutagen; and
(c) cultivating the cutting to produce a mutated plant.

22. The method of claim 21 wherein the mutagen is a chemical mutagen.

23. The method of claim 21 wherein the mutagen is radiation.

24. The method of claim 23 wherein the radiation is gamma radiation.

25. (canceled)

26. A part of a plant as in claim 35, selected from the group consisting of a flower, cutting, a pollen grain, an ovule, a cell, a seed or an embryo.

27. A method for producing an interspecific hybrid Euphorbia plant comprising:
(a) providing a first plant which is a Euphorbia pulcherrima plant and a second plant which is a species of Euphorbia that is not Euphorbia pulcherrima;
(b) pollinating a flower of the second plant with pollen from the first plant or a flower of the first plant with pollen from the second plant in a manner which permits formation of an embryo in at least one ovule of the pollinated plant;
(c) cutting the embryo; and
(d) culturing the cut embryo by placing the cut embryo in contact with culture medium to permit growth of the embryo to thereby produce a primary plant.

28. A plant produced according to the method of claim 27.

29. A part of a plant produced according to the method of claim 27.

30. The method of claim 21 wherein the mutated plant or a portion of the mutated plant is propagated by:
(a) obtaining a bract from the mutated plant;
(b) placing the bract in a solution capable of disinfesting the bract;
(c) washing the bract; and
(d) cultivating the bract to thereby produce a propagated mutated plant.

31. The method of claim 1 wherein the primary plant, or a portion of the primary plant, may be propagated by:
(a) obtaining a bract from the primary plant;
(b) placing the bract in a solution capable of disinfesting the bract;
(c) washing the bract; and
(d) cultivating the bract to thereby propagate the primary plant.

32. A method for propagating a Euphorbia plant comprising:
(a) obtaining a bract from the plant;
(b) placing the bract in a solution capable of disinfesting the bract;
(c) washing the bract; and
(d) cultivating the bract to thereby produce a propagated mutated plant.

33. The method of claim 32 wherein the solution capable of disinfesting the bract is bleach.

34. The method of claims 33 wherein the bleach is used at a concentration of between 1% and 3%.

35. A plant or part of a plant produced according to the method of claim 1.

36. The method of claim 22 wherein the mutated plant or a portion of the mutated plant is propagated by:
(a) obtaining a bract from the mutated plant;
(b) placing the bract in a solution capable of disinfesting the bract;
(c) washing the bract; and
(d) cultivating the bract to thereby produce a propagated mutated plant.

37. The method of claim 23 wherein the mutated plant or a portion of the mutated plant is propagated by:
(a) obtaining a bract from the mutated plant;
(b) placing the bract in a solution capable of disinfesting the bract;
(c) washing the bract; and
(d) cultivating the bract to thereby produce a propagated mutated plant.

38. The method of claim 24 wherein the mutated plant or a portion of the mutated plant is propagated by:
(a) obtaining a bract from the mutated plant;
(b) placing the bract in a solution capable of disinfesting the bract;
(c) washing the bract; and
(d) cultivating the bract to thereby produce a propagated mutated plant.

39. The method of claim 30 wherein the solution capable of disinfesting the bract is bleach.

40. The method of claim 31 wherein the solution capable of disinfesting the bract is bleach.
